(19) United States
(12) Patent Application Publication
(10) Pub. No.: US 2024/0181199 A1
(43) Pub. Date: Jun. 6, 2024

(54) SYSTEM FOR PROVIDING BIO-RESONANCE THERAPY AND USE THEREOF

(71) Applicant: Wave Force Electronics Inc., Mill Bay (CA)

(72) Inventors: Stephen Davis, Mill Bay (CA); Steven King, Mill Bay (CA)

(73) Assignee: Wave Force Electronics Inc., Mill Bay (CA)

(21) Appl. No.: 18/528,653

(22) Filed: Dec. 4, 2023

(30) Foreign Application Priority Data
Dec. 2, 2022 (CA) 3184042

(51) Int. Cl. A61M 16/14; A61M 16/00; A61M 16/10

(52) U.S. Cl. CPC A61M 16/147; A61M 16/0066; A61M 16/105; A61M 2205/3584

(57) ABSTRACT

An imprinting system is provided for imprinting a therapeutic material, the imprinting system comprising an imprinting device and a computing device, wherein the imprinting device includes: a plastic polymeric housing which includes a lid; a box which is housed in the housing and includes a lid, a bottom which includes a multiplicity of apertures, and sides, which define an interior; a multiplicity of light emitting diodes which are mounted in the multiplicity of apertures; a printed circuit board which is housed in the housing and is in electric communication with the multiplicity of light emitting diodes, and wherein the computing device includes a user interface, a memory and a processor, the computing device in wired or wireless communication with the printed circuit board, the memory including at least one digitized frequency.

### CROSS REFERENCE TO RELATED APPLICATIONS

This application claims priority to Canadian Patent Application Serial No. 3,184,042, filed Dec. 2, 2022, entitled SYSTEM FOR PROVIDING BIO-RESONANCE THERAPY AND USE THEREOF. The above-identified priority patent application is incorporated herein by reference in its entirety.

### FIELD

The present technology is a system for imprinting a substance or material with at least one frequency and delivering the at least one frequency to a user. More specifically it is a system comprising an integrated capture-imprinter device and computing device for capturing at least one frequency, storing at least one frequency in a database and imprinting at least one substance or material with at least one frequency from the database, then providing the at least one frequency to the user.

### BACKGROUND

While several methods currently exist for applying specific electromagnetic frequencies to substances, largely they consist of mere copies of single frequencies from a sample substance to a carrier. While effective, these methods are lacking in depth and scope of application, as well as limitations on the number of remedies available and how those remedies can be combined.

For example, EP2418000 discloses a method for converting and transmitting the energy information signal. It provides for the reception of a super weak polarized energy information signal from the signal source and the use of the said signal to act on a biological or other object. The energy information signal is converted into an information wave bundle and brought into the coherent state by passing it through at least one crystal arranged between two polarizers, so that the converted signal subsequently acts on the object, including for the purpose of direct action or for the purpose of mediated action using an intermediate carrier which has been subjected to the action of the converted energy information signal. According to individual configurations, the action on an object takes place in that it experiences the action of a carrier which has been exposed to the action of the converted energy information signal. According to individual configurations, the source of the energy information signal is the biological object, in particular the body or a substance from the following group: histological preparation on the object glass, whole blood, blood plasma or biological fluid: urine, saliva, sperm, tear, sweat, skin print and/or a piece of resect or its imprint on any material. The converter contains contact 1, cup (container) 2 for receiving the object for exposure, upper base plate 3, support 4, film holder 5, the upper polarizing film 6, quartz plate 7, quartz holder 8, the second film holder 9, the second support 10, Adjustment driver 11, the lower polarizing film 12, the lower ring 13 of the film holder 12, lower base plate 14, the third support 15, damper (4 pcs.) 16, support surface for the source of the energy information signal. Thus, the effective method and device for amplifying, converting and inverting the signal of both biological objects as well as drugs, foods and carriers have been developed. As a result of exposure to the converted energy information signal, the biological object has a 25% smaller general biological index (ABI), a 20% smaller photon index (FI) and a threefold increase in the adaptation reserve (AR). The product has at least 25% increased characteristic effectiveness indicators. The reliability of the information signal amplification is increased, the workflow in connection with applying the information to the carrier is simplified, the duration of treatment of various pathologies, including cancer, is shortened. This is an overly complex system that converts and inverts the energy information signal.

WO2012160549 discloses homeopathic combinations, carriers imprinted with the bio-resonance of said combinations and articles of manufacture comprising the same. More specifically it is directed to the use of bio-resonance therapy for the prevention, protection-from, and treatment of various diseases and conditions, including radiation, cancer, metabolic disorders, neurodegenerative disorders, and cellular metabolic disorders. This system transfers the frequencies from the homeopathic formulation to a carrier by way of vibration.

Imprinting devices are disclosed at WAVE TRANSFER and MEDICUP are used for analog duplication and storage of bioinformation (https://www.magnetotherapy.de/our-products/devices-for-therapists/wave-transferc/wave-transfer.html). One MEDICUP attached to the WAVE TRANSFER device for input and another MEDICUP is attached to the WAVE TRANSFER for output. The substrate to be duplicated is put into the input MEDICUP. An aqueous solution is put in the output MEDICUP and transfer is started. This system can transfer the frequency spectra of one or more ampules/substrates simultaneously or successively to a single carrier medium. The disclosed use of these imprinting devices is different to what is contemplated in the present technology and does not include a means for delivery of the bio-resonance therapy.

WO2011062518 discloses method ensures that the energy information signals are transmitted to a first intermediate vector, then information is transmitted to an input section of a receptor, and signals are obtained at a receiving side of the receptor by transmitting the signals to a second intermediate (the last) vector, so as to apply the signals to any economic fields. As for the remote transmission of an energy information signal from animate and inanimate objects, energy information signals from an original object pass through at least one crystal between two polarizers and are converted into an information wave beam and turned into coherent state, the converted signals are recorded onto the first intermediate vector, enter into a transceiver and are transmitted together with electromagnetic wave signals on cable and wireless communication networks (lines), signals received are then recorded on the second vector, and specific energy information signals of the original object are read from the received signals. The specific energy information signals of the original object are read from the second vector by a biophysical apparatus from a BRT or VRT group. The converter comprises a contactor (1), shell (container) (2) used for super weak energy information signals to act on the object, an upper base (3), a first pillar (4), a sheet support (5), an upper polarizer (6), a quartz sheet (7), a quartz support (8), a second sheet support (9), a second pillar (10), an adjustment handle (11), a lower polarizer (12), a ring beam (13) of the sheet support, a lower base (14), a third pillar (15), a damper (16) and a platform for the source of the energy information signal. High-efficiency transmission of the energy information signals in a certain distance is ensured and the technology is simplified. When the invention is applied to medical science, the process time is shortened, and functional capabilities for diagnoses of various symptoms and determination of promising treatment directions are expanded. This is an overly complex system.

DE102011109338 discloses a device for storing electromagnetic energy and signals, for example from biological systems. The device can record electrical signals and, if necessary, modify it again. This device is provided with a planar substrate having a first side and a second side, wherein on the first side at least one first arrangement is provided, the at least one first cavity (2), at least one first initiator (4) and at least one first device for storing electromagnetic energy (3), one end of the inducer (4) being connected to the electromagnetic energy storage device (3) and the other end of the inducer (4) being the free end in, under or adjacent to first cavity (2) is arranged. Preferred materials for the construction of the device are disclosed. This system does not include a means for delivery of the signals.

DE4447328 discloses a pharmaceutical applicator in the form of an electromagnetic storage device exhibits the bio-resonance spectrum of a pharmaceutical which is suitable of acting directly on a biological receptor system. Preparation of a pharmaceutical applicator comprises generating the bio-resonance signal of the pharmaceutical with the aid of a frequency generator of frequency range 1-150 kHz which continuously produces the bio-resonance signal, amplifying the signal by a predetermined factor and then storing it on an electromagnetic storage device. This system transfers the frequencies from the pharmaceutical formulation and not to the pharmaceutical formulation, thus the user is not gaining an additional advantage.

AT1358 discloses a device for delivering homeopathic information with a conventional device which emits vibrations, the device is connected according to the inventive method to at least one electrical conductor which supplies the device with homeopathic information which the device emits as a vibration. This system transfers the frequencies from the homeopathic formulation and not to the homeopathic formulation, thus the user is not gaining an additional advantage.

US Patent Application Publication No. 20130135981 discloses a method comprised of transferring energy information signals onto a first intermediate carrier, with subsequent transmission of information to a receiver input and taking off the signal at the reception side of the receiver by transferring the signal onto a second intermediate carrier, for use in any area of the economy. For remote transmission of an energy information signal from animate and inanimate objects, the energy information signal of an original object is converted to an information-wave beam and brought to a coherent state by passing the beam through a crystal located between two polarizers. The converted signal is recorded onto the first intermediate carrier, entered into a transceiver and transmitted over wire and wireless communication networks together with an electromagnetic wave signal, with subsequent recording of the received signal onto the second carrier and reading off the second carrier the energy information signal peculiar to the original object. This system is overly complex.

According to Journal of Saudi Chemical Society Volume 22, Issue 7, November 2018, Pages 855-875 “The components of essential oil are lipophilic terpenoids, phenylpropanoids, or short-chain aliphatic hydrocarbon derivatives. They have the molecular weight less than 300. Most essential oils are obtained by hydro-distillation method. The stability of essential oils depends on several internal factors and external factors that lead to some chemical reaction. The internal factors include chemical structure and impurities present in the essential oil. The external factors include presence of oxygen, exposure to light, humidity and temperature. The stability of essential oil against degradation depends on molecular structure of the compound. Auto-oxidation leads to a breakdown of the chemical structure or modification of the chemical structure when essential oils are exposed to oxygen. The essential oils obtained from pine and turpentine mainly containing unsaturated mono- and sesqui-terpenes are not stable upon storage. The polysaturated hydrocarbons containing conjugated double bonds could form several stabilized radicals, which leads to oxidative degradation. Light illumination on an essential oil leads to considerable changes in its composition and structure. This is due to various kinds of reaction mechanism. For instance, monoterpenes undergo rapid degradation in the presence of light. In lemon oil it has been observed that the amounts of geranial, terpinolene, and c-terpinene are decreased with a rise in p-cymene. In sweet fennel oil, the trans-anethole was completely oxidized to anisaldehyde or isomerized to cis-anethole after 2 months of storage at room temperature under light. Under ultraviolet or visible light, essential oil undergoes auto-oxidation. Visible light also promotes the cis-trans isomerization.” Hence, the use of light to imprint essential oils brings with it special considerations and requirements, as does exposure to oxygen.

What is needed is a bio-resonance system that provides an additional treatment modality in a selected therapeutic treatment. More specifically, a system that includes a library of frequencies, an imprinter and a fluid communication means is needed. The library would preferably be an extensive and ever-growing repository of frequencies derived from biological sources. It would be preferable if the system included a user interface which allowed a provider to select one of more frequencies. It would be preferable if the selected frequencies were complementary to the therapeutic remedy being imprinted. It would be still further preferable if the imprinted therapeutic remedy was dispensed with a diffuser or other means of fluidly communicating the imprinted therapeutic remedy to a client. It would be further preferable if the system was designed for imprinting one or more essential oils and providing a therapy with the imprinted essential oil.

### SUMMARY

The present technology is a bio-resonance system that provides an additional treatment modality in a selected therapeutic treatment. More specifically, it is a system that includes a library of frequencies, an imprinter and a fluid communication means. The library is an extensive and ever-growing repository of frequencies derived from biological sources. The system includes a user interface which allowed provider to select one of more frequencies. The selected frequencies are complementary to the therapeutic remedy being imprinted. The imprinted therapeutic remedy is dispensed with a diffuser or other means of fluidly communicating the imprinted therapeutic remedy to a client. The system is designed for imprinting one or more essential oils and providing a therapy with the imprinted essential oil.

This technology addresses the need for a fully programmable bio-resonance therapy (BRT) device that is capable of applying a large range of digitized frequencies from a proprietary database into any substance or object that can be placed within, or otherwise connected to, the BRT imprint device.

In other embodiments, this functionality may also be applied through a plate or via a handheld instrument. More particularly, the invention relates to a bio-resonance imprinting system comprising an interface providing access to a secure database of digitized frequencies that may be selected individually and/or as part of a pre-set package, an imprinting device such as an enclosure, receptacle, or external handheld instrument, and a substance or object to receive the frequency imprint for the benefit of the user.

In one embodiment, an imprinting system is provided for imprinting a therapeutic product, the imprinting system comprising an imprinting device and a computing device, wherein the imprinting device includes: a plastic polymeric housing which includes a lid; a box which is housed in the housing and includes a lid, a bottom which includes a multiplicity of apertures, and sides, which define an interior; a multiplicity of light emitting diodes which are mounted in the multiplicity of apertures; a printed circuit board which is housed in the housing and is in electric communication with the multiplicity of light emitting diodes, and wherein the computing device includes a user interface, a memory and a processor, the computing device in wired or wireless communication with the printed circuit board, the memory including at least one digitized frequency.

The imprinting system may further comprise a cloud storage device, the cloud storage device retaining a library of frequencies, the cloud storage device in wired or wireless communication with the computing device.

The imprinting system may further comprise a capture device, the capture device in wired or wireless communication with the computing device.

In the imprinting system, the imprinting device may further include a fan and holder for an oxygen absorbing cartridge, the fan disposed to urge an ambient air flow over the holder and into the box.

In the imprinting system, the memory may be configured to receive at least one digitized frequency from the cloud storage device and to instruct the processor to instruct the printed circuit board to control an imprinting with at least one of the multiplicity of light emitting diodes.

In another embodiment, a method of imprinting a therapeutic product is provided, the method comprising: a) a user selecting a system comprising an imprinting device and a computing device, wherein the imprinting device includes: a plastic polymeric housing which includes a lid; a box which is housed in the housing and includes a lid, a bottom which includes a multiplicity of apertures, and sides, which define an interior; a multiplicity of light emitting diodes which are mounted in the multiplicity of apertures; a printed circuit board which is housed in the housing and is in electric communication with the multiplicity of light emitting diodes, and wherein the computing device includes a user interface, a memory and a processor, the computing device in wired or wireless communication with the printed circuit board, the memory including at least one digitized frequency; b) the user placing the therapeutic product in the box and closing the lid; c) the user, via the user interface, selecting a frequency for imprinting and initiating the imprinting of the therapeutic product with the frequency; d) and the imprinting device imprinting the therapeutic product.

In the method, the system may further include a cloud storage device which includes a library of digitized frequencies and the method may further comprise the computing device receiving the digitized frequency from the cloud storage device prior to imprinting.

In the method, the therapeutic product may be an essential oil.

In the method, the imprinting device may further include a fan, a holder and an oxygen absorbing cartridge which is retained in the holder, and the method may further comprise the fan urging an ambient airflow over the oxygen absorbing cartridge prior to imprinting.

In another embodiment, an integrated capturer-imprinter is provided for use with a computing unit, for capturing a frequency of a therapeutic material and for imprinting a product to provide a therapeutic product, the integrated capturer-imprinter comprising: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes.

In the integrated capturer-imprinter, the integrated capture-imprinter may further include a fan and holder for an oxygen absorbing cartridge, the fan disposed to urge an ambient air flow over the holder and into the box.

In another embodiment, a method of imprinting a therapeutic product is provided, the method comprising: a) a user selecting a system comprising a computing unit and an integrated capturer-imprinter device, wherein the capturer-imprinter device includes: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes, and wherein the computing unit includes a user interface, a memory and a processor, the computing unit in wired or wireless communication with the printed circuit board; b) the user placing a frequency emitting material in the box and closing the lid; the computing unit collecting and retaining a digitized copy of a frequency from the frequency emitting material; c) the user placing therapeutic product in the box and closing the lid; d) the user, via the user interface, selecting the digitized copy of the frequency for imprinting and initiating the imprinting of the therapeutic product with the frequency; e) and the integrated capturer-imprinter imprinting the therapeutic product.

In the method, the system may further include a cloud storage device which includes a library of digitized frequencies and the method further comprises the computing unit sending the digitized frequency from the cloud storage device.

In the method, the integrated capturer-imprinter device may further include a fan, a holder and an oxygen absorbing cartridge which is retained in the holder, and the method may further comprise the fan urging an ambient airflow over the oxygen absorbing cartridge prior to imprinting.

In another embodiment, a system for capturing a frequency of a therapeutic material and for imprinting a product to provide a therapeutic product is provided, the system comprising a computing unit and an integrated capturer-imprinter, the integrated capturer-imprinter comprising: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes, wherein the computing unit includes a user interface, a memory and a processor, the computing unit in wired or wireless communication with the printed circuit board, the memory including at least one digitized frequency.

The system may further comprise a cloud storage device, the cloud storage device retaining a library of frequencies, the cloud storage device in wired or wireless communication with the computing unit.

In the system, the integrated capturer-imprinter may further include a fan and holder for an oxygen absorbing cartridge, the fan disposed to urge an ambient air flow over the holder and into the box.

In the system, the memory may be configured to receive at least one digitized frequency from the cloud storage device and to instruct the processor to instruct the printed circuit board to control an imprinting with at least one of the multiplicity of light emitting diodes.

### BRIEF DESCRIPTION OF THE DRAWINGS

FIG. 1 is a schematic of the bio-resonance system of the present technology.

FIG. 2 is a schematic of the imprinting device of the system of FIG. 1.

FIG. 3 is a block diagram of the electronics of the system.

FIG. 4 is a block diagram of the method of imprinting and delivering a bio-resonance treatment.

FIG. 5 is a schematic of an integrated capture-imprinting device.

FIG. 6 is a schematic of the calibration circuit for the capture mode of the device of FIG. 5.

### DESCRIPTION

Except as otherwise expressly provided, the following rules of interpretation apply to this specification (written description and claims): (a) all words used herein shall be construed to be of such gender or number (singular or plural) as the circumstances require; (b) the singular terms “a”, “an”, and “the”, as used in the specification and the appended claims include plural references unless the context clearly dictates otherwise; (c) the antecedent term “about” applied to a recited range or value denotes an approximation within the deviation in the range or value known or expected in the art from the measurements method; (d) the words “herein”, “hereby”, “hereof”, “hereto”, “hereinbefore”, and “hereinafter”, and words of similar import, refer to this specification in its entirety and not to any particular paragraph, claim or other subdivision, unless otherwise specified; (e) descriptive headings are for convenience only and shall not control or affect the meaning or construction of any part of the specification; and (f) “or” and “any” are not exclusive and “include” and “including” are not limiting. Further, the terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller sub ranges are also included. The upper and lower limits of these smaller ranges are also included therein, subject to any specifically excluded limit in the stated range.

Unless defined otherwise, all technical and scientific terms used herein have the same meaning as commonly understood by one of ordinary skill in the relevant art. Although any methods and materials similar or equivalent to those described herein can also be used, the acceptable methods and materials are now described.

### Definitions

Fluid communication—in the context of the present technology, fluid communication includes movement of a gas or gases from a location to another location and movement of a liquid or liquids from a location to another location. The fluid may be directed for example, as a flow or may be diffused.

Fluid communicator—in the context of the present technology, a fluid communicator moves gas or gases from one location to another or moves a liquid or liquids from one location to another. Movement may be a flow or may be a diffusion.

Direct frequency—in the context of the present technology, a direct frequency is one that is collected from an object, imprinted and used in its same form, as opposed to an inverse frequency, which is inverted prior to use.

Library of frequencies—in the context of the present technology, a library of frequencies is a collection of digitized frequencies ranging from 0.1 Hertz to 1017 Hertz.

Computing unit—in the context of the present technology a computing unit may be a separate device or may be integrated into another device.

### DETAILED DESCRIPTION

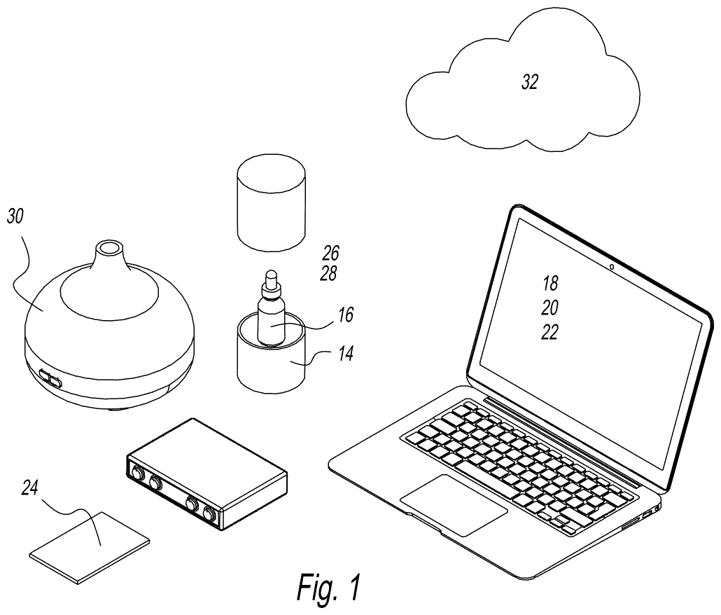

A bio-resonance system, generally referred to as 12 is shown in FIG. 1. The system includes: a capture chamber 14, in which a frequency emitting material 16 is held during capture; a computing device 18 which includes a memory 20 and a processor 22, wherein the memory 20 includes instructions for instructing the processor 22 to process the frequencies that are captured, the memory 20 including a library of digitized frequencies, which in one embodiment are direct frequencies and in another embodiment, are other than direct frequencies; and an imprinting device 24. The system 12 may further or alternatively include cloud-based storage 32 for the library of frequencies. The cloud-based storage 32 is in wired or wireless communication with the computing device 18. The library is proprietary and is ever-growing. By using a secure, cloud-based, database of digitized frequencies, a much larger range of options is available relative to a standard therapeutic remedy imprint made by a practitioner. The imprinting is conducted directly on a diffuser 30 and the liquid to be diffused is added after the diffuser 30 is imprinted. In one embodiment the liquid to be diffused is water.

In another embodiment, at least one ampule 26 which contains a therapeutic compound or therapeutic formulation 28 is for placing in the imprinting device.

In one embodiment, the frequency emitting material 16 is a biological material. In another embodiment the frequency emitting material 16 is an organic compound, organic formulation or organic mixture. In another embodiment, the frequency emitting material 16 is a drug. In another embodiment, the frequency emitting material 16 is a therapeutic compound, or therapeutic material or therapeutic mixture. In one embodiment, the frequency emitting material 16 is an essential oil or a mixture of essential oils. In a preferred embodiment, the therapeutic compound or therapeutic formulation 28 in the ampule 26 is water or an essential oil or essential oils and the frequency emitting material 16 is an organic compound, organic formulation, organic mixture, a drug, a therapeutic compound, therapeutic material or therapeutic mixture. In an alternative embodiment, the ampule 26 is replaced with any material to be imprinted, for example, but not limited to, a container, more specifically a diffuser, a wearable, more specifically a watch band, a band, a ring, an article of clothing, footwear or a consumable.

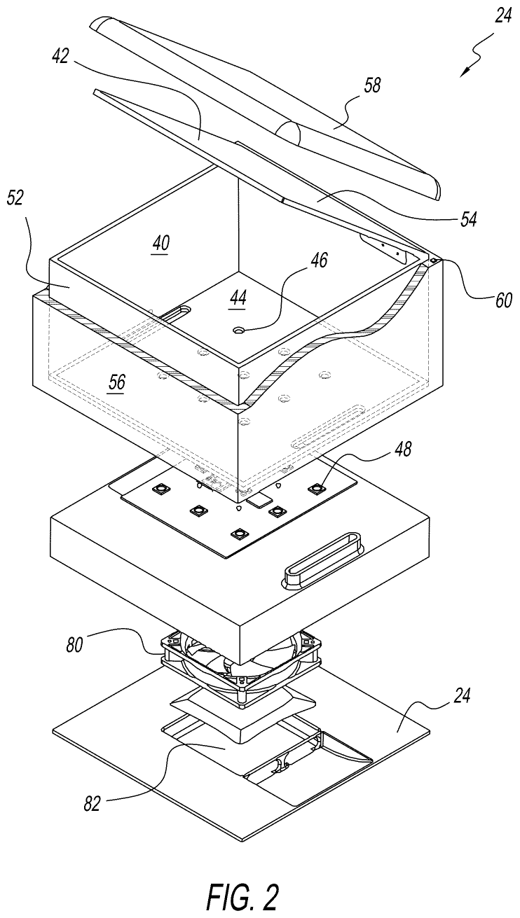

As shown in FIG. 2, in one embodiment, the imprinting device 24 includes a box 40 with a lid 42 and a bottom 44 which includes a multiplicity of apertures 46. A multiplicity of light emitting diodes 48 (LEDs) are aligned with the multiplicity of apertures 46. The LEDs 48 include infrared (IR), near-infrared (NIR), ultraviolet (UV) or visible light or any combination thereof. The box 40 has four fixed sides 52 and a top 54 that is affixed to the underside of the lid 42 which forms a conductive seal when pressed closed.

In an alternative embodiment, the multiplicity of apertures 46 are in a side 52. The box 40 is preferably made of aluminum, copper, steel, or any electrically conductible metal or combination thereof or is coated or plated with an electrically conductive metal. The inner surface has a matte finish. A plastic polymeric housing 56 surrounds the box 40 and also has a lid 58. A locking mechanism 60, for example, but not limited to a pin switch-type mechanism or magnetic proximity sensor, promotes secure closure of the lid 58. The plastic polymeric housing 56 functions as an opaque covering for the box 40 and electrical insulation against the voltage levels applied to the box 40. The light emitting diodes 48 are housed in the plastic polymeric housing 56 in addition to electrical circuitry. The plastic polymeric housing 56 can be in any one of a range of shapes, including but not limited to a cube, rectangle, a cylinder, or any other geometric shape of suitable interior volume. The plastic polymeric housing 56 protects the process from being interfered with or disrupted and provides a uniform environment for the materials being imprinted. The box 40 and the plastic polymeric housing 56 reduce or eliminate light-mediated degradation of the material being imprinted. Further, they also reduce or eliminate any extraneous electromagnetic signals being introduced. The housing 56 retains a fan 80 and holder 82 for an oxygen absorbing cartridge, such that the fan 80 urges the ambient air over/through the oxygen absorbing cartridge. The oxygen absorbing cartridge reduces or eliminates oxidation of the material being imprinted that could be caused by the LEDs 48 or during e-field imprinting, should there be ambient oxygen.

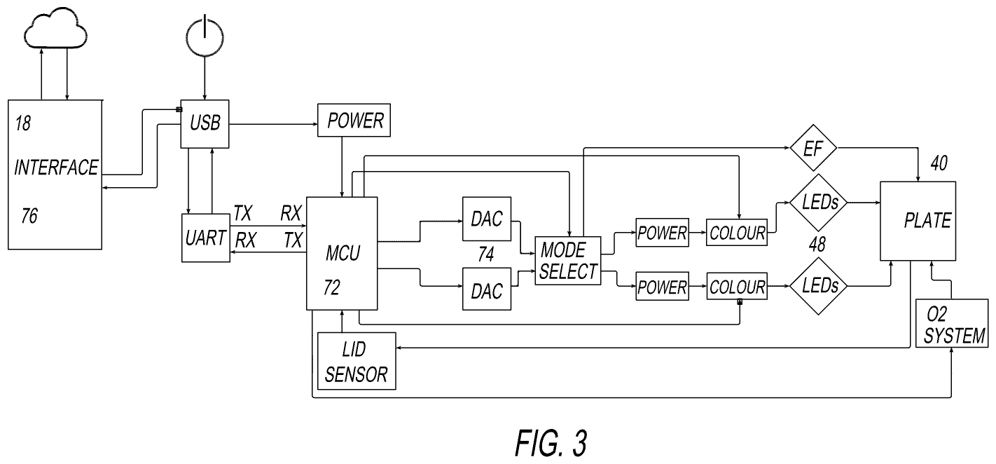

As shown in FIG. 3, the light emitting diodes 48 are in electrical communication with a printed circuit board 72 and are affixed to the printed circuit board 72. The printed circuit board 72 includes at least two separate output channels 74 that can be used simultaneously and independently and which provide imprinting using different imprinting modalities and modes. The printed circuit board 72 is in electronic communication with a user interface 76. In one embodiment, light emitting diodes 48 are used to imprint and the different imprinting modes include different wavelengths, different brightness levels, signal duration and splitting of the frequencies delivered between different light emitting diodes. To be clear, the light spectrum of the light emitting diodes 48 is not the frequency used for imprinting, but rather, it is the pulsing of the light emitting diodes 48 that is the frequency used for imprinting.

In another embodiment, e-field imprinting is used through a direct application of voltage potential to the box 40.

The printed circuit board 72 uses a microcontroller and associated components to receive a signal from the interface device, which may be received via a wired means such as USB cable or wireless such as Bluetooth® and converts it to a suitably formed analog output. This output is transmitted to the object placed within the receptacle via coded pulses of light. In an alternative embodiment, the output is pulsations in the electrical field created within the receptacle, depending on the selected frequencies and the object or substance being imprinted.

In an alternative embodiment, the imprinting device 24 is a specialized plate, open receptacle, or other form of handheld accessory device for objects that will not fit within the box 40.

In an alternative embodiment, the user interface 76 is part of the computing device 18 which is in electronic communication with the printed circuit board 72 of the imprinting device 24. Communication may be wired or wireless. The computing device 18 may be integrated into the housing 56. The memory 20 or the cloud-based storage 32 or both (generically referred to as electronic data storage device) features several broad categories of intent, which may include examples such as sleep, calm, or focus. These broad categories may include one or more premade packages, as well individual frequencies that may be selected.

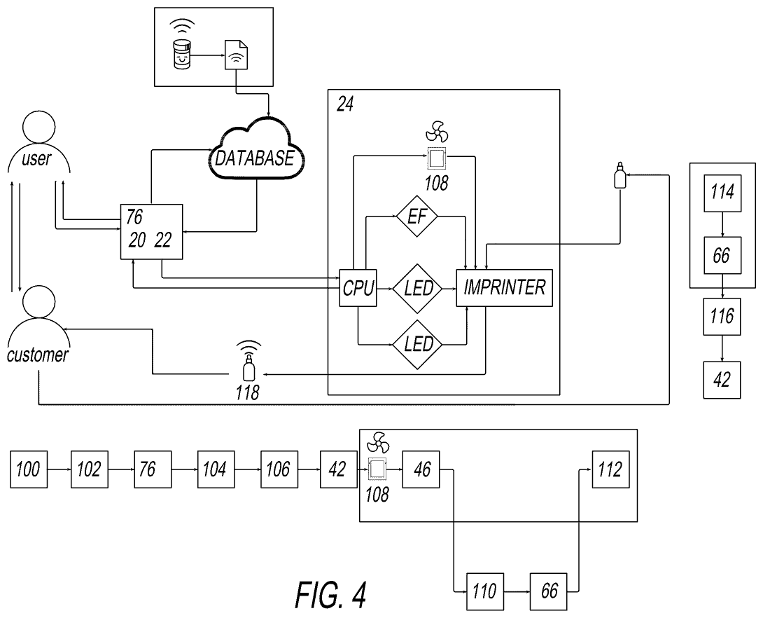

As shown in FIG. 4, a customer selects 100 a physical product to purchase to which one or more bio-resonance therapy frequencies can be applied for an enhanced effect. The store clerk, technician, or practitioner presents 102 a selection of available bio-resonance frequencies and/or frequency packages to the customer, preferably with a standard recommendation for their chosen product. This is similar to a restaurant server suggesting specific wines and/or appetizers to go with a chosen entree. Each product comes with a suggestion of a standard pairing of one or more frequencies, although the customer is free to choose anything that would not directly hinder the effects of either the selected product or the applied frequencies. The selection process involves the clerk navigating into the appropriate section on the user interface 76 and selecting a combination of packages and/or individual frequencies and dragging them to the package builder section of the user interface 76. This process may or may not also include inputting the chosen product the customer is purchasing, to help guide the frequency selection process by both suggesting appropriate packages and narrowing the selection to compatible frequencies for that product. Once the customer or clerk has chosen their bio-resonance therapy frequencies, the clerk places 104 the physical product to be imprinted into the imprinting device 24 and holds 106 the lid 42 closed. The fan is actuated 108, urging the ambient air over/through the oxygen absorbing cartridge and urging at least partially oxygen depleted air through the apertures 46. The clerk then pushes 110 the appropriate button on the user interface 66 to begin imprinting, and the selected frequencies are emitted 112 in the imprinting device 24. After the imprinting process is accomplished successfully, the printed circuit board sends a completion signal 114 to the user interface 66. Once the imprinting process has concluded, the clerk opens 116 the lid 42 and removes 118 the product to hand to the customer. The process may be repeated for any additional products the customer may be purchasing, if desired. The user interface 76, under control of the processor 22 returns to the main screen after a successful imprint. A history function is accessible for a short period of time after an imprint. The memory 20 has instructions for the processor 22 to communicate with the printed circuit board to conduct a frequency clearing function thereafter. Note that this function is repeated when a new imprint is about to be made.

Imprinting may be light, electrical signal or a combination thereof and including frequency delivery split amongst multiple modalities depending on a variety of factors including the materials being imprinted and the number or type(s) of frequencies being applied.

In one embodiment, there are nine LEDs which are split into alternating arrays of 5 and 4 lights respectively: XOX, OXO AND XOX, which can be controlled either separately or in sync (selectable by the firmware code). This allows for running either the same frequency for each array, different frequencies for each array (to imprint multiple items simultaneously), different colours for each array, or colour shifting for each array over the span of each imprint, which has approximately a two second duration.

In another embodiment the signal alternates between in phase and 180° out of phase, between each array to create an optical standing wave where an increase in light intensity of one array causing a corresponding reduction in light intensity of the other.

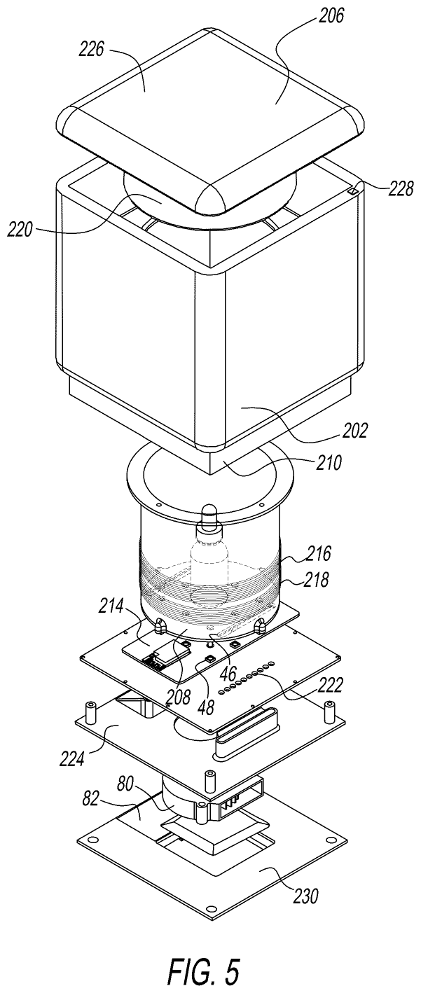

In an alternative embodiment, as shown in FIG. 5, an integrated capture chamber-imprinter device, generally referred to as 200 is provided. An enclosure 202 has plastic polymeric sides 204 and lid 206 and a metal imprint plate 208 for a base. The base 208 is made of, for example, but not limited to aluminum, copper or steel. The metal imprint plate 208 is capable of having a time-varying electrical potential applied to it which corresponds to a target frequency from the frequency database. The metal imprint plate 208 has apertures 46 with an insulating air gap, which pulsed light from an array of LED 48 modules may shine through.

A Faraday shield 210 envelops the enclosure 202, the printed circuit board 212, the imprinting components 214 and the two magnetic coils of the capture component, a pickup coil 216 and an exciter coil 218. The Faraday shield 210 protects these from ambient electromagnetic fields. The Faraday shield 210 is made of a conductive material, for example, but not limited to aluminum, copper or steel. The Faraday shield 210 includes a door 220 which is aligned with the lid 206 of the enclosure 202. In one embodiment, the enclosure 202 does not have a lid 206. Apertures 222 in the Faraday shield 210 are at least one of no more than 4 millimeters in diameter or contain multiple 90° turns to prevent electromagnetic field intrusion. The enclosure 202 is supported by the Faraday shield 210 as is the housing 224. The housing 224 is made of a plastic polymeric material and has a lid 226. A locking mechanism 228, for example, but not limited to a pin switch-type mechanism or magnetic proximity sensor, promotes secure closure of the lid 226. The plastic polymeric housing 224 functions as an opaque covering for the enclosure 202 and electrical insulation against the voltage levels applied within the enclosure 202. As described in relation to the imprinting device 24, the light emitting diodes 48 and electrical circuitry are housed in the plastic polymeric housing 224. The plastic polymeric housing 224 can be in any one of a range of shapes, including but not limited to a cube, rectangle, a cylinder, or any other geometric shape of suitable interior volume. The plastic polymeric housing 224 protects the process from being interfered with or disrupted and provides a uniform environment for the materials being imprinted. The enclosure 202 and the plastic polymeric housing 224 reduce or eliminate light-mediated degradation of the material being imprinted. Further, they also reduce or eliminate any extraneous electromagnetic signals being introduced.

A bottom plate 230 retains a fan 80 and holder 82 for an oxygen absorbing cartridge, such that the fan 80 urges the ambient air over/through the oxygen absorbing cartridge. The oxygen absorbing cartridge reduces or eliminates oxidation of the material being imprinted that could be caused by the LEDs 48 or during e-field imprinting, should there be ambient oxygen. The bottom plate 230 is releasably retained on the housing 224. The circuitry for imprinting is as described in relation to FIG. 3 above.

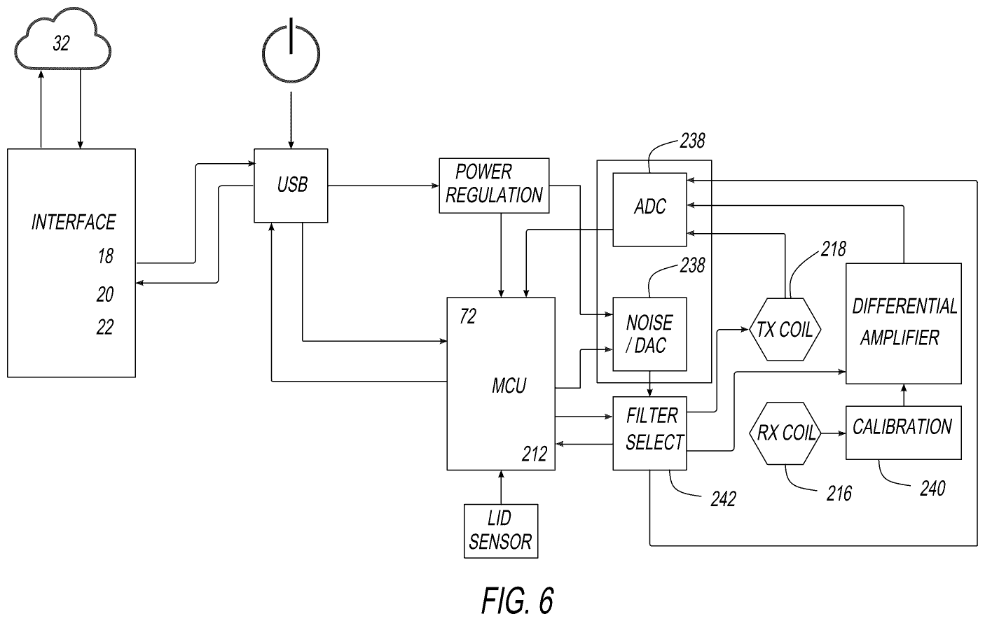

The calibration circuit is shown in FIG. 6. In use, during capture, a driving signal of Gaussian/white noise is injected into the exciter coil 218, and the pickup coil 216 is tuned to have zero output when there is nothing placed within the field. This is accomplished by injecting a white noise signal from a signal generator 238 into the exciter coil 218 and measuring the received waveform on the pickup coil 216. These signals are analyzed by the printed circuit board 212 and the tuning of the pickup coil 216 is adjusted electronically until there is no difference. Sequential digital potentiometers 240 allow for extremely precise automatic calibration of the recorded electromagnetic fields. A bandpass filter 242 is used to limit the frequencies.

When an object or substance is placed within the field after previously being adjusted to achieve null input, the output signal shall change slightly, as detected by a differential amplifier. This signal is then amplified and digitally processed to obtain a clear signal which corresponds to the sample substance using the components as follows: a computing device 18 which includes a memory 20 and a processor 22, wherein the memory 20 includes instructions for instructing the processor 22 to process the frequencies that are captured, the memory 20 including a library of digitized frequencies, which in one embodiment are direct frequencies and in another embodiment, are other than direct frequencies; and an imprinting device 24. The system 12 may further or alternatively include cloud-based storage 32 for the library of frequencies. The cloud-based storage 32 is in wired or wireless communication with the computing device 18. The library is proprietary and is ever-growing. By using a secure, cloud-based, database of digitized frequencies, a much larger range of options is available relative to a standard therapeutic remedy imprint made by a practitioner. In one embodiment, the Gaussian noise circuit uses the method of using a reverse-biased transistor forced into avalanche mode to produce pure white noise. In another embodiment, a DAC, DSP, or other signal generation method may also be used to generate pseudo-random noise, specific pulsations, sweeps, or other signal patterns for driving the exciter/TX coil. In order to eliminate unwanted EMF/RF signals, such as those from nearby devices or power lines, the bandpass filter 242 is selectable to anywhere within 1-50 KHz; the electronics capabilities allow for up to 4 different ranges for either applying different noise ranges to different samples, or sequentially applying different noise ranges and then digitally comparing, merging, averaging, or otherwise processing the resultant signals. The Gaussian noise circuit, before the bandpass filter stage, is also be used during the imprinting process to drive the LEDs for frequency clearing.

The details of the capturing process are as follows: During the capture process, a filtered noise signal is injected into the exciter coil 218 and that same signal is measured by the microcontroller of the printed circuit board 72. The pickup coil 216 has a time-varying voltage induced in it, which is modified slightly by the presence of an object or substance within the field. Both the exciter coil waveform signal and the received pickup coil signal are passed through a differential amplifier which removes the excitation signal and leaves, nominally, the electromagnetic signature of the target substance. This signal is extremely weak and requires amplification before it can be properly processed, analyzed, or stored in analog form. During a capture process, the microcontroller records both the exciter coil signal and the pickup coil signal through onboard analog to digital converters (ADCs) where the absolute values can be compared in real-time. This provides a variety of options for signal processing, both for initial calibration and output processing. The input signals can be both compared directly as digitized versions of the analog values, or a more complex process involving Fourier transforms can be performed. By multiplexing through a variety of bandpass filter arrays, multiple signals can be recorded for comparison or processing. This may be advantageous if certain substances respond better to certain ranges than others, whether due to molecular weight, magnetic or paramagnetic properties, or some other factor.

The recorded waveform may be processed in a number of ways. If the resultant differential signals differ depending on the noise signal input, experimentation will be needed to determine the optimal range and what the contributing factor is, as mentioned above. If the resultant differential signals are the same, perhaps with some small difference or distortion at the edges, then an averaging or summing of the signals may be used. Each wave may be analyzed with a Fourier transform to break down the waveform components, which may allow for a more detailed comparison of the different frequency ranges. Similarly, cither an averaging or a summing operation can be performed with the resultant data. Finally, multiple passes can be performed in a very short period of time, to get a large enough sampling of the same data to compare and/or average to determine that the final signal is accurate. A multitude of noise sources may be utilized sequentially or together to derive the clearest output signal.

The details of the imprinting are as outlined above as is the circuitry and components. In an alternative embodiment, the magnetic coils 216, 218 used for the frequency capture process can also be used for imprinting, alone or in combination with the LED 48 arrays or the e-field method.

In additional embodiments of the technology the technician or practitioner uses frequencies and products specified in a work order, purchase order, or prescription to be delivered or shipped to an end user or supplied for retail sale, but otherwise the process is identical.

By using a proprietary application connected to a cloud-based database, licensing and access control are easily implemented to protect the frequency database, imprint device, and all other aspects of the system from misuse.

This could include, but would not be limited to, abusing the frequencies by misapplying them in a way that would be detrimental to the user, limiting use to prevent unauthorized mass-imprinting of products, or anything else not specifically designated as an approved use in an agreement with the user.

For a particular group of users, such as multiple clerks at distributed retail locations of a specific company, offices within a medical or wellness facility, or technicians at a pharmacy, dispensary or manufacturing facility, access may be limited to only the frequencies that correspond to their product line. Additionally, access may be granted based on licensing of frequency packages on a purchase or subscription access basis.

Further security measures may be implemented based on factors including matching IP address or similar identifying information, to protect against theft or unauthorized use of an imprint device outside of an approved licensed location.

Further, an encrypted hardware identification and handshake protocol ensures that no third party interference is taking place to intercept or disrupt communications, and that no unauthorized activities are taking place for the purpose of falsifying data communications within the system, extracting, retrieving or intercepting data sent to or from any part of the system, and to prevent cloned or spoofed communications to or from any aspect of the system, including a counterfeit application, an inline device, or a mechanism intended to replicate the imprinter device itself. Each imprinter device has a unique identifier built into the hardware which cannot be modified or spoofed. This identifier be linked to the specific account it is linked with for database access, in the case of a retail chain this could be in the form of store23@domain.com, to tie it to a specific geolocation.

While example embodiments have been described in connection with what is presently considered to be an example of a possible most practical and/or suitable embodiment, it is to be understood that the descriptions are not to be limited to the disclosed embodiments, but on the contrary, is intended to cover various modifications and equivalent arrangements included within the spirit and scope of the example embodiment. Those skilled in the art will recognize or be able to ascertain using no more than routine experimentation, many equivalents to the specific example embodiments specifically described herein. Such equivalents are intended to be encompassed in the scope of the claims, if appended hereto or subsequently filed.

1. An imprinting system for imprinting a therapeutic product, the imprinting system comprising an imprinting device and a computing unit, wherein the imprinting device includes: a plastic polymeric housing which includes a lid; a box which is housed in the housing and includes a lid, a bottom which includes a multiplicity of apertures, and sides, which define an interior; a multiplicity of light emitting diodes which are mounted in the multiplicity of apertures; a printed circuit board which is housed in the housing and is in electric communication with the multiplicity of light emitting diodes, and wherein the computing unit includes a user interface, a memory and a processor, the computing device in wired or wireless communication with the printed circuit board, the memory including at least one digitized frequency.

2. The imprinting system of claim 1, further comprising a cloud storage device, the cloud storage device retaining a library of frequencies, the cloud storage device in wired or wireless communication with the computing unit.

3. The imprinting system of claim 2, further comprising a capture device, the capture device in wired or wireless communication with the computing unit.

4. The imprinting system of claim 3, wherein the imprinting device further includes a fan and holder for an oxygen absorbing cartridge, the fan disposed to urge an ambient air flow over the holder and into the box.

5. The imprinting system of claim 4, wherein the memory is configured to receive at least one digitized frequency from the cloud storage device and to instruct the processor to instruct the printed circuit board to control an imprinting with at least one of the multiplicity of light emitting diodes.

6. A method of imprinting a therapeutic product, the method comprising: a) a user selecting a system comprising an imprinting device and a computing device, wherein the imprinting device includes: a plastic polymeric housing which includes a lid; a box which is housed in the housing and includes a lid, a bottom which includes a multiplicity of apertures, and sides, which define an interior; a multiplicity of light emitting diodes which are mounted in the multiplicity of apertures; a printed circuit board which is housed in the housing and is in electric communication with the multiplicity of light emitting diodes, and wherein the computing device includes a user interface, a memory and a processor, the computing device in wired or wireless communication with the printed circuit board, the memory including at least one digitized frequency; b) the user placing the therapeutic product in the box and closing the lid; c) the user, via the user interface, selecting a frequency for imprinting and initiating the imprinting of the therapeutic product with the frequency; d) and the imprinting device imprinting the therapeutic product.

7. The method of claim 6, wherein the system further includes a cloud storage device which includes a library of digitized frequencies and the method further comprises the computing device receiving the digitized frequency from the cloud storage device prior to imprinting.

8. The method of claim 7, wherein the therapeutic product is an essential oil.

9. The method of claim 8, wherein the imprinting device further includes a fan, a holder and an oxygen absorbing cartridge which is retained in the holder, and the method further comprises the fan urging an ambient airflow over the oxygen absorbing cartridge prior to imprinting.

10. An integrated capturer-imprinter for use with a computing unit, for capturing a frequency of a therapeutic material and for imprinting a product to provide a therapeutic product, the integrated capturer-imprinter comprising: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes.

11. The integrated capturer-imprinter of claim 10, wherein the integrated capture-imprinter further includes a fan and holder for an oxygen absorbing cartridge, the fan disposed to urge an ambient air flow over the holder and into the box.

12. A method of imprinting a therapeutic product, the method comprising: a) a user selecting a system comprising a computing unit and an integrated capturer-imprinter device, wherein the capturer-imprinter device includes: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes, and wherein the computing unit includes a user interface, a memory and a processor, the computing unit in wired or wireless communication with the printed circuit board; b) the user placing a frequency emitting material in the box and closing the lid; the computing unit collecting and retaining a digitized copy of a frequency from the frequency emitting material; c) the user placing therapeutic product in the box and closing the lid; d) the user, via the user interface, selecting the digitized copy of the frequency for imprinting and initiating the imprinting of the therapeutic product with the frequency; e) and the integrated capturer-imprinter imprinting the therapeutic product.

13. The method of claim 12, wherein the system further includes a cloud storage device which includes a library of digitized frequencies and the method further comprises the computing unit sending the digitized frequency from the cloud storage device.

14. The method of claim 13, wherein the integrated capturer-imprinter device further includes a fan, a holder and an oxygen absorbing cartridge which is retained in the holder, and the method further comprises the fan urging an ambient airflow over the oxygen absorbing cartridge prior to imprinting.

15. A system for capturing a frequency of a therapeutic material and for imprinting a product to provide a therapeutic product, the system comprising a computing unit and an integrated capturer-imprinter, the integrated capturer-imprinter comprising: a plastic polymeric housing which includes a lid; a metal box which is housed in the housing and includes a lid, a bottom which includes a multiplicity of apertures, and sides, which define an interior; a pickup coil and an exciter coil, both which are housed in the metal box; a multiplicity of light emitting diodes which are mounted in the multiplicity of apertures; and a printed circuit board which is housed in the housing and is in electric communication with the pickup coil, the exciter coil and the multiplicity of light emitting diodes, wherein the computing unit includes a user interface, a memory and a processor, the computing unit in wired or wireless communication with the printed circuit board, the memory including at least one digitized frequency.

16. The system of claim 15, further comprising a cloud storage device, the cloud storage device retaining a library of frequencies, the cloud storage device in wired or wireless communication with the computing unit.

17. The system of claim 16, wherein the integrated capturer-imprinter further includes a fan and holder for an oxygen absorbing cartridge, the fan disposed to urge an ambient air flow over the holder and into the box.

18. The system of claim 17, wherein the memory is configured to receive at least one digitized frequency from the cloud storage device and to instruct the processor to instruct the printed circuit board to control an imprinting with at least one of the multiplicity of light emitting diodes.
